(19) United States
(12) Patent Application Publication
(10) Pub. No.: US 2009/0004284 A1
(43) Pub. Date: Jan. 1, 2009

(54) Controlled release tamsulosin hydrochloride formulation

(75) Inventors: Xiu Xiu Cheng, Weston, FL (US); Xiufang Cheng, Weston, FL (US)

Correspondence Address: HEDMAN & COSTIGAN P.C., 1185 AVENUE OF THE AMERICAS, NEW YORK, NY, 10036, US

(73) Assignee: Watson Pharmaceuticals, Inc.

(21) Appl. No.: 11/821,990

(22) Filed: Jun. 26, 2007

(51) Int. Cl. A61K 9/14; A61K 31/18

(52) U.S. Cl. 424/497; 424/490; 514/603

(57) ABSTRACT

The present invention relates to a controlled release pellet of tamsulosin and its pharmaceutically acceptable salts that comprise an inert starting seed or core.

### FIELD OF THE INVENTION

The present invention relates to the field of oral pharmaceutical dosage forms with controlled release of an active ingredient. The oral pharmaceutical dosage form can be a tablet, capsule or suspension. In one embodiment the oral pharmaceutical dosage form employs mini tablets, pellets or coated granules that can be directly administered by suspending them in a liquid, sprinkling them onto food or incorporating them into a tablet, capsule or suspension. Preferably the oral pharmaceutical dosage form employs a drug core loaded with a low percentage of active ingredient, such as an α₁ₐ-selective alpha blocker, preferably tamsulosin or a pharmaceutically acceptable salt such as a hydrochloride salt. Tamsulosin and it pharmaceutically acceptable salts are disclosed in U.S. Pat. No. 4,868,216 and U.S. Pat. No. 4,703,063; both of which are incorporated herein by reference.

### BACKGROUND OF THE INVENTION

A constant time controlled and/or a pH specific release of the active component of an orally administered drug is advantageous in medical treatment.

U.S. Pat. No. 4,772,475 discloses methods of preparing tamsulosin formulations comprising granulating individual units or single units of tamsulosin. Additional wet granulation techniques for formulating tamsulosin are disclosed in U.S. Pat. No. 6,861,070; both of which are incorporated herein by reference. Other formulations of tamsulosin are disclosed in U.S. Pat. Nos. 7,018,658; 6,932,983; 6,923,988; 6,800,668; 6,720,001; 6,645,528; 6,569,463; 6,383,471; and 6,294,192.

A commercially available form of tamsulosin hydrochloride is sold by Boehringer Ingelheim under the tradename FLOMAX®. FLOMAX® is a capsule formulation that is administered once daily for the treatment of benign prostatic hyperplasia (BPH). According to the package insert for FLOMAX® each capsule contains tamsulosin hydrochloride, methacrylic acid copolymer, microcrystalline cellulose, triacetin, polysorbate 80, sodium lauryl sulfate, calcium stearate, talc and various coloring agents.

### SUMMARY OF THE INVENTION

An object of the present invention is to provide a stable oral dosage form that is easy to manufacture, and that can be administered once daily.

The present invention accomplishes this object and other objectives that will be readily apparent to individuals of ordinary skill in the art by preparing a controlled or sustained release core. The core can be prepared by blending or granulating the drug with a binder and filler or by layering the drug onto an inert starting seed. The core may be further coated with a release controlling membrane.

In one embodiment, the core of the present invention is prepared by spheronizing the drug with a binder, preferably a combination of a water soluble binder and a water insoluble binder or by layering the drug and binder onto a starting seed. The core is then coated with a release controlling membrane that is preferably a combination of a pH dependent material, such as an enteric polymer and a water insoluble polymer.

In another embodiment, the core can be prepared by mixing the drug with a binder, as described above, and optionally other excipients and compressing the mixture into tablets or mini tablets. The tablets or mini tablets are then coated with a release controlling membrane as described above.

In a still further embodiment, the present invention employs controlled release pellets comprising:
a) an inert core;
b) a drug layer applied to the inert core; and
c) a controlled release membrane surrounding the drug layer.

The pellets can be mixed with conventional tabletting excipients and compressed into a tablet or loaded into a capsule for oral administration.

The present invention also relates to a method of producing the pellets or beads.

Additionally, the present invention can include an immediate release form of the drug. The immediate release form of the drug can be obtained by applying a rapidly releasing layer of the drug to the final dosage form or incorporating an immediate release pellet or granule into the tablet, capsule or suspension.

### DETAILED DESCRIPTION OF THE INVENTION

The following detailed description is for illustration only and is not intended to limit the scope of the invention.

The pharmaceutically active ingredient employed in the present invention is as an α₁ₐ-selective alpha blocker, preferably tamsulosin or a pharmaceutically acceptable salt such as a hydrochloride salt. Tamsulosin and it pharmaceutically acceptable salts are disclosed in U.S. Pat. No. 4,868,216 and U.S. Pat. No. 4,703,063; both of which are incorporated herein by reference. The tamsulosin or its pharmaceutically acceptable salts may be in its racemic form or a pure enantiomer. The most preferred salt is tamsulosin hydrochloride.

The drug is then mixed with a binder and optionally other excipients to prepare a core. The core can be prepared by wet or dry granulation followed by compression into tablets, mini tablets or by extrusion spheronization.

In a preferred embodiment, the core of the present invention is prepared by applying the drug and binder onto an inert core or inert seed. The inert core must be of sufficient density and strength to enable it to undergo coating in a fluidized bed process. The inert cores of the present invention should have a diameter less than 15 mesh and preferably less than 25 mesh. In a preferred embodiment the inert cores should have a diameter ranging from about 20 to 80 mesh, preferably 25 to 60 mesh and most preferred 30 to 35 mesh. Suitable inert cores are sugar seeds (or non-pareils) and microcrystalline cellulose spheres, which are commercially available from FMC Corporation under the trade name CELPHERE®. Polyproplyene or silicon dioxide starting seeds could also be employed in the present invention

The binder employed in the present invention can be any type of binder commonly known in the art. Examples of some of the preferred binding agents are polyvinyl pyrrolidone, hydroxyethyl cellulose, hydroxypropyl cellulose, hydroxypropyl methylcellulose, polyacrylate, ethylcellulose, cellulose acetate, cellulose acetate butyrate, polyvinyl alcohol, polyvinyl acetate, sodium alginate, starch, clays, naturally occurring gums or mixtures of the foregoing. In one embodiment of the present invention, the binder is a combination of a water soluble or rapidly dispersible material, such as hydroxypropyl cellulose and a water insoluble material such as ethylcellulose.

As used herein the term water insoluble material includes polymeric materials that are slightly permeable to water.

The drug may be applied to the inert core by any conventional techniques known in the industry, such as, pan coating, roto-granulation or fluidized bed coating. During such coating operations the drug and binder are dispersed or dissolved in an organic or aqueous solvent, which may also contain other conventional excipients.

One optional excipient that may be used to prepare the core is a surfactant. If a surfactant is employed, it can be any type of surfactant commonly known in the art such as a fatty acid, a chelating agent, a bile salt or mixtures thereof. Examples of some preferred surfactants are fatty acids such as capric acid, oleic acid and their monoglycerides, especially alkyl sulfates, such as sodium lauryl sulfate, sodium dodecyl sulfate and polysorbate 80; chelating agents such as citric acid and phytic acid. The preferred surfactant used herein is sodium lauryl sulphate.

The core may also be prepared using other conventional ingredients such as fillers, lubricants, glidants, and anti-foaming agents. In one embodiment, the core is free (i.e., does not contain) of any pH dependent or enteric material.

In a preferred embodiment of the present invention, the core comprises the following ingredients:

TABLE I
Drug Core

| Ingredients | Preferred | Most Preferred |
|---|---|---|
| Filler | 70-99.5% | 85-99% |
| Drug | 0.01-10% | 0.05-5% |
| Binder | 0.01-15% | 0.05-10% |
| Surfactant | 0.01-10% | 0.05-5% |

Once the core is prepared, a controlled release membrane is applied to the core. The controlled release membrane is applied so that it prevents or retards the release of the drug from the core. The controlled release coat is preferably comprised of a combination of polymeric film forming polymers and may optionally contain conventional processing aids such as emulsifiers, plasticizers, surfactants, lubricants or channeling agents.

The film forming polymers suitable for use in the controlled release coating are the water insoluble polymeric coating agents such as such as, ethylcellulose, cellulose acetate, cellulose propionate (lower, medium or higher molecular weight), cellulose acetate propionate, cellulose acetate butyrate, triacetate, cellulose tricetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene acrylate), poly(ethylene), poly(ethylene) low density, poly(ethylene) high density, poly(propylene), poly(ethylene terephthalate), poly(vinyl isobutyl ether), poly(vinyl acetate), poly(vinyl chloride), chitin, chitosan, poly(anhydrides), poly(lactic acid), ploy(glycolic acid), poly(ortho esters), poly(lactide co-glycollide), poly(hydroxy butyrate) or polyurethane or a mixture thereof.

The controlled release coating should also contain a pore forming agent, which is a material that will dissolve or hydrate in gastrointestinal fluid and create channels or pores to aid in the release of the drug. Some common pore forming agents are water soluble materials such as sugars (i.e., sucrose, lactose fructose) and salts. In one embodiment of the present invention the controlled release membrane employs a pH dependent pore forming agent. A pH dependent pore forming agent is a material that dissolves only at certain pHs. An example of a suitable pH dependent pore forming agent is an enteric polymer. Enteric polymers are well known in the art and any suitable enteric polymers may be used. Preferably, the enteric polymer is selected from cellulose acetate phthalate, hydroxypropyl methylcellulose phthalate, polyvinylacetate phthalate, methacrylic acid copolymer, shellac, hydroxypropyl methylcellulose succinate, cellulose acetate trimellitate, hypromellose phthalate and mixtures of any of the foregoing. The preferred pore forming agent is hypromellose phthalate.

The controlled release coating may be built up by applying a plurality of coats of polymer solution or suspension to the drug core as hereinafter described. The membrane solution or suspension contains the polymer(s) dissolved or suspended, respectively, in a suitable aqueous or organic solvent or mixture of solvents, optionally in the presence of other conventional excipients.

The controlled release coating solution or suspension may be applied to the active cores in a conventional coating pan as indicated or, alternatively, using an automated system such as a CF granulator, for example a FREUND CF® granulator, a GLATT® fluidized bed processor, a modified ACCELA-COTA® or other suitably automated bead coating equipment.

Suitable emulsifiers that can be used in the present invention may include, but are not limited to, phospholipids, polysorbate, propylene glycol, poloxamer, glyceryl monostearate, other pharmaceutical emulsifiers and/or mixtures thereof.

Suitable surfactants that may optionally be used in the present invention are sodium lauryl sulfate, sodium taurocholate or a polysorbate.

The controlled release coating may optionally include a plasticizing agent. Plasticizers are used to increase the resiliency of the finished product from cracking and fracturing. Suitable plasticizing agents include polyethylene glycol, propylene glycol, glycerol, triacetin, dimethyl phthalate, diethyl phthalate, dibutyl phthalate, dibutyl sebacate, triethyl citrate, acetyltributyl citrate, tributyl citrate, triethyl acetyl citrate, castor oil, poloxamers and varying percentages of acetylated monoglycerides. The preferred plasticizer is acetyltributyl citrate.

Suitable lubricants possess anti-sticking or anti-tacking properties. Suitable lubricants used in preparing solid dosage forms may include talc, stearic acid, magnesium stearate, glyceryl monostearate, sodium stearyl fumerate, hydrogenated oils, polyethylene glycols and sodium stearate. A particularly preferred lubricant is talc.

In a preferred embodiment of the present invention, the controlled release coating comprises the following ingredients:

TABLE II
Controlled Release Coating

| Ingredients | Preferred | Most Preferred |
|---|---|---|
| Water insoluble | 10-50% | 20-40% |
| polymer | | |
| Pore forming agent | 10-50% | 20-40% |
| Plasticizer | 0-25% | 5-15% |
| Lubricating agent | 0-60% | 0-50% |

The above weight percentages are based on the total weight of the newly formed controlled release coating layer.

The oral pharmaceutical dosage form prepared in accordance with the present invention should exhibit the following dissolution profile when tested in a USP Type 2 apparatus, at 50 rpm, 37° C. in TWEEN® 80/1.2 buffer for 2 hours followed by a phosphate buffer medium with a pH of 7.2 for 8 hours.

TABLE III

| Time | Preferred | Most Preferred |
|---|---|---|
| 2 hours | 0-40% | 0-25% |
| 4 hours | 40-95% | 70-90% |
| 6 hours | 70-99% | 80-98% |
| 8 hours | 80-99.9% | 90-99.9% |
| 10 hours | NTL 80% | NTL 90% |

NTL = not less than

The oral pharmaceutical dosage form prepared in accordance with the present invention should exhibit the following dissolution profile when tested in a USP Type 2 apparatus, at 50 rpm, 37° C. and in pH 4.5 buffer.

TABLE IV

| Time | Preferred | Most Preferred |
|---|---|---|
| 2 hours | 0-30% | 0-15% |
| 4 hours | 10-50% | 15-40% |
| 6 hours | 15-60% | 20-50% |
| 8 hours | 20-70% | 30-60% |
| 12 hours | 25-80% | 35-70% |

The oral pharmaceutical dosage form prepared in accordance with the present invention should exhibit the following dissolution profile when tested in a USP Type 2 apparatus, at 50 rpm, 37° C. and in distilled water.

TABLE V

| Time | Preferred | Most Preferred |
|---|---|---|
| 2 hours | 0-30% | 0-15% |
| 4 hours | 5-40% | 10-35% |
| 6 hours | 15-60% | 20-45% |
| 8 hours | 20-70% | 25-50% |
| 12 hours | 25-80% | 30-60% |

The oral pharmaceutical dosage form prepared in accordance with the present invention should exhibit the following dissolution profile when tested in a USP Type 2 apparatus, at 50 rpm, 37° C. and in pH 6.8.

TABLE VI

| Time | Preferred | Most Preferred |
|---|---|---|
| 2 hours | 0-70% | 0-60% |
| 4 hours | 50-90% | 60-85% |
| 6 hours | 60-98% | 70-95% |
| 8 hours | 70-99% | 85-98% |
| 12 hours | NTL 75% | NTL 85% |

If an immediate release dose of the active ingredients is desired for the final dosage form, the tablet, capsule or suspension may further comprise a therapeutically effective amount of the drug which can be mixed into the tabletting excipients or with the controlled release coated pellets prior to encapsulation. In a preferred embodiment, the immediate release amount of the drug is provided by adding active drug pellets or pellets which have not been coated with the controlled release coating to the controlled release coated pellets prior to encapsulation or tabletting.

### DESCRIPTION OF THE PREFERRED EMBODIMENT

The present invention will be further illustrated by the following examples.

### EXAMPLE I

A controlled release tamsulosin hydrochloride capsules in accordance with the present invention is prepared as follows.

### Stage I Drug Layering Process

8.00 g of tamsulosin is dissolved in a mixture of 600 g of purified water and 2400 g of ethyl alcohol (SDA 3A 190 proof) using a mechanical stirrer until a clear solution is obtained. 4.00 g of hydroxypropyl cellulose (Klucel EF) is then added into the solution until completely dissolved. Then 60.00 g of ethyl cellulose (Ethylcellulose NF, 7 mPas) is added to the solution and mixing is continued until it is completely dissolved. Next, 40.00 g of sodium lauryl sulphate is added to the solution and mixing is continued until it is completely dissolved. Once the drug is added, the suspension should be constantly stirred until the spraying process is completed to avoid settling.

3,888 g of sugar spheres NF 30/35 are placed into a fluidized bed coater. The product air temperature of the coater should be between 30 and 45° C. prior to loading the sugar spheres into the fluidized bed coater. The drug suspension prepared above is sprayed onto the sugar seeds using the following conditions:

TABLE VII

| | |
|---|---|
| Nozzle tip diameter | 1.2 mm |
| Screen Size | 50 mesh |
| Shaking interval | 5 min |
| Shaking Duration | 3 sec |
| Atomization Pressure | 2.5 bar |
| Inlet Air Temperature | 30° C. |
| Tubing Size | 24 mm |

Once the drug suspension has been consumed the pellets are dried until the loss on drying (LOD) is less than 2%. Finally, the pellets are screened using 20 mesh and 50 mesh screens. The pellets between the 20 and 50 mesh are collected.

Stage II Polymer Coating process for Tamsulosin Hydrochloride ER Pellets
A controlled release coating is prepared with the following composition:

TABLE VIII

| | Amount Per Batch |
|---|---|
| Ingredients | (kg) |
| Tamsulosin HCl Active Pellets | 3.6 |
| Hypromellose Phthalate, NF 31% (HP-55) | 0.400 |
| Ethylcellulose, NF 7 cps | 0.400 |
| Acetyltributyl citrate, NF | 0.160 |
| Talc USP | 0.480 |

The controlled release coating is prepared by dissolving 0.160 kg of the acetyltributyl citrate, NF, into a mixture of 2.112 kg of purified water and 8.448 kg of ethyl alcohol and stirred until the solution is clear using a mechanical stirrer. 0.400 kg of Hypromellose Phthalate (NF 31% (HP-55)) is added to the solution and stirred until it is completely dissolved. Next, 0.400 kg of Ethylcellulose (NF 7 cps) is added to the solution and stirred until it is completely dissolved. Then, 0.480 kg of talc is added to the solution and stirred until it is completely dissolved. The solution is then applied to the active or drug layered pellets prepared above in Stage I using a bottom spray fluidized bed coater with the same parameters as described for the application of the drug layer in Stage I.

After the controlled release solution has been consumed the pellets are dried until the LOD is less than 2%. Finally, pellets are screened through 20 and 50 mesh screens and the pellets between the 20 and 50 mesh screens are collected.

Pellets may be dusted with an anti-sticking agent which may include, but would not be limited to talc or silicon dioxide (commercially available as Aerosil®).

### Stage III Encapsulation of the Tamsulosin Controlled Release Pellets

A capsule in accordance with the present invention is prepared with the following composition:

TABLE IX

| Ingredients | Amount per Batch (g) |
|---|---|
| Tamsulosin Controlled | 123.2 |
| Release Pellets | |
| (Stage II) | |
| Size “1”, Hard Gelatin | 26.64 |
| Capsule | |

### EXAMPLE II

A controlled release tamsulosin hydrochloride capsules in accordance with the present invention is prepared as follows.

### Stage I Drug Layering Process

8.00 g of tamsulosin is dissolved in a mixture of 600 g of purified water and 2400 g of ethyl alcohol (SDA 3A 190 proof) using a mechanical stirrer until a clear solution is obtained. 4.00 g of hydroxypropyl cellulose (Klucel EF) is then added into the solution until completely dissolved. Then 60.00 g of ethyl cellulose (Ethylcellulose NF, 7 mPas) is added to the solution and mixing is continued until it is completely dissolved. Once the drug is added, the suspension should be constantly stirred until the spraying process is completed to avoid settling.

3,928 g of sugar spheres NF 30/35 are placed into a fluidized bed coater. The product air temperature of the coater should be between 30 and 45° C. prior to loading the sugar spheres into the fluidized bed coater. The drug suspension prepared above is sprayed onto the sugar seeds in accordance with the parameters set forth in Example I above.

Once the drug suspension has been consumed the pellets are dried until the loss on drying (LOD) is less than 2%. Finally, the pellets are screened using 20 mesh and 50 mesh screens. The pellets between the 20 and 50 mesh are collected.

Stage II Polymer Coating process for Tamsulosin Hydrochloride ER Pellets
A controlled release coating is prepared with the following composition:

TABLE X

| | Amount Per Batch |
|---|---|
| Ingredients | (kg) |
| Tamsulosin HCl Active Pellets | 3.6 |
| Hypromellose Phthalate, NF 31% (HP-55) | 0.400 |
| Ethylcellulose, NF 7 cps | 0.400 |
| Acetyltributyl citrate, NF | 0.160 |
| Talc USP | 0.480 |

The controlled release coating is prepared by dissolving 0.160 kg of the acetyltributyl citrate, NF, into a mixture of 2.112 kg of purified water and 8.448 kg of ethyl alcohol and stirred until the solution is clear using a mechanical stirrer. 0.400 kg of Hypromellose Phthalate (NF 31% (HP-55)) is added to the solution and stirred until it is completely dissolved. Next, 0.400 kg of Ethylcellulose (NF 7 cps) is added to the solution and stirred until it is completely dissolved. Then, 0.480 kg of talc is added to the solution and stirred until it is completely dissolved. The solution is then applied to the active or drug layered pellets prepared above in Stage I using a bottom spray fluidized bed coater with the same parameters as described for the application of the drug layer in Stage I.

After the controlled release solution has been consumed the pellets are dried until the LOD is less than 2%. Finally, pellets are screened through 20 and 50 mesh screens and the pellets between the 20 and 50 mesh screens are collected.

Pellets may be dusted with an anti-sticking agent which may include, but would not be limited to talc or silicon dioxide (commercially available as Aerosil®).

### Stage III Encapsulation of the Tamsulosin Controlled Release Pellets

A capsule in accordance with the present invention is prepared with the following composition:

TABLE XI

| Ingredients | Amount per Batch (g) |
|---|---|
| Tamsulosin Controlled | 109.2 |
| Release Pellets | |
| (Stage II) | |
| Size “1”, Hard Gelatin | 24.61 |
| Capsule | |

The oral pharmaceutical dosage form prepared in accordance with the present invention exhibited the following dissolution release percentage when tested in a USP Type 2 apparatus, at 50 rpm, 37° C. and in TWEEN® 80/1.2 buffer for 2 hours followed by a phosphate buffer medium with a pH of 7.2 for 8 hours.

TABLE XII

| Time (Hrs) | Example I | Example II |
|---|---|---|
| 0.5 | 0 | 0 |
| 1 | 0 | 0 |
| 2 | 10 | 7 |
| 2.5 | 37 | 27 |
| 3 | 66 | 53 |
| 4 | 89 | 80 |
| 5 | 94 | 90 |
| 6 | 96 | 95 |
| 8 | 97 | 98 |
| 10 | 98 | 99 |

The oral pharmaceutical dosage form prepared in accordance with the present invention exhibited the following dissolution release percentage when tested in a USP Type 2 apparatus, at 50 rpm, 37° C. and in pH 6.8 buffer.

TABLE XIII

| Time (Hrs) | Example I | Example II |
|---|---|---|
| 0.5 | 0 | 0 |
| 1 | 13 | 7 |
| 2 | 29 | 23 |
| 3 | 51 | 48 |
| 4 | 65 | 63 |
| 5 | 76 | 73 |
| 6 | 88 | 85 |
| 8 | 95 | 93 |
| 12 | 99 | 100 |

The oral pharmaceutical dosage form prepared in accordance with the present invention exhibited the following dissolution release percentage when tested in a USP Type 2 apparatus, at 50 rpm, 37° C. and in ph 4.5.

TABLE XIV

| Time (Hrs) | Example I | Example II |
|---|---|---|
| 0.5 | 0 | 0 |
| 1 | 0 | 0 |
| 2 | 0 | 0 |
| 3 | 10 | 7 |
| 4 | 22 | 16 |
| 5 | 28 | 23 |
| 6 | 34 | 32 |
| 8 | 37 | 38 |
| 12 | 39 | 45 |

The oral pharmaceutical dosage form prepared in accordance with the present invention should exhibit the following dissolution profile when tested in a USP Type 2 apparatus, at 50 rpm, 37° C. and in distilled water.

TABLE XV

| Time (Hrs) | Example I | Example II |
|---|---|---|
| 0.5 | 0 | 0 |
| 1 | 0 | 0 |
| 2 | 0 | 0 |
| 3 | 11 | 8 |
| 4 | 20 | 16 |
| 5 | 26 | 22 |
| 6 | 33 | 29 |
| 8 | 36 | 34 |
| 12 | 37 | 40 |

The Product of Example I was compared to commercially available FLOMAX® under fasting conditions in 12 patients in accordance with FDA bioequivalence testing guidelines. The results are report in Table XVI.

TABLE XVI

| | Example I | FLOMAX ® | EX I/Flomax ® | |
|---|---|---|---|---|
| | LS Means | LS Mean | Ratio | 90% C.I. |
| Cmax | 9.6465 | 9.7830 | 87.25 | 77.9-97.7 |
| AUC 0~t | 12.1851 | 12.3276 | 86.71 | 77.0-97.7 |
| AUC 0~inf | 12.2232 | 12.3630 | 86.95 | 77.1-98.1 |
| Tmax | 4.96 | 4.71 | 105.31 | 100.0-110.7 |

The Product of Example I was compared to commercially available FLOMAX® under fed conditions in 12 patients in accordance with FDA bioequivalence testing guidelines. The results are report in Table XVII.

TABLE XVII

| | Example I | FLOMAX ® | | |
|---|---|---|---|---|
| | LS Means | LS Mean | EX I/Flomax ® Ratio | 90% C.I. |
| Cmax | 8.9059 | 8.9025 | 100.34 | 86.8-115.9 |
| AUC 0~t | 11.9781 | 11.9664 | 101.18 | 90.8-112.8 |
| AUC | 12.0118 | 12.0266 | 98.53 | 87.8-110.6 |
| 0~inf | | | | |
| Tmax | 9.79 | 11.96 | 81.88 | 52.7-111.0 |

The Product of Example II was compared to commercially available FLOMAX® under fasting conditions in 12 patients in accordance with FDA bioequivalence testing guidelines. The results are report in Table XVIII.

TABLE XVIII

| | Example I | FLOMAX ® | EX I/ | |
|---|---|---|---|---|
| | LS Means | LS Mean | Flomax ® Ratio | 90% C.I. |
| Cmax | 9.5872 | 9.7830 | 82.21 | 73.4-92.1 |
| AUC 0~t | 12.2144 | 12.3276 | 89.29 | 79.3-100.6 |
| AUC | 12.2599 | 12.3630 | 90.20 | 79.9-101.8 |
| 0~inf | | | | |
| Tmax | 4.92 | 4.71 | 104.42 | 99.1-109.38 |

The Product of Example II was compared to commercially available FLOMAX® under fed conditions in 12 patients in accordance with FDA bioequivalence testing guidelines. The results are report in Table XIX.

TABLE XIX

| | Example I | FLOMAX ® | | |
|---|---|---|---|---|
| | LS Means | LS Mean | EX I/Flomax ® Ratio | 90% C.I. |
| Cmax | 8.8522 | 8.9025 | 95.09 | 83.2-110.0 |
| AUC 0~t | 11.9420 | 11.9664 | 97.59 | 87.5-108.8 |
| AUC | 11.9845 | 12.0266 | 95.88 | 85.4-107.6 |
| 0~inf | | | | |
| Tmax | 9.67 | 11.96 | 80.84 | 51.7-110.0 |

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

We claim:

1. A controlled release pellet comprising:
a) an inert core;
b) a drug layer applied to the inert core comprising:
i) tamsulosin hydrochloride,
ii) a binder, and
iii) optionally a surfactant; and
c) a controlled release coating surrounding the drug layer.

2. The controlled release pellet as defined in claim 1, wherein the inert core is a sugar seed.

3. The controlled release pellet as defined in claim 1, wherein the binder is selected from the group consisting of polyvinyl pyrrolidone, hydroxyethyl cellulose, hydroxypropyl cellulose, hydroxypropyl methylcellulose, polyacrylate, ethylcellulose, cellulose acetate, cellulose acetate butyrate, polyvinyl alcohol, polyvinyl acetate, sodium alginate, starch, clays, naturally occurring gums or mixtures thereof.

4. The controlled release pellet as defined in claim 3, wherein the binder is a mixture of a water soluble material and a water insoluble material.

5. The controlled release pellet as defined in claim 4, wherein the water insoluble material is ethylcellulose and the water soluble material is hydroxypropyl cellulose.

6. The controlled release pellet as defined in claim 1, wherein the drug layer further comprises a surfactant.

7. The controlled release pellet as defined in claim 6, wherein the surfactant is selected from the group consisting of fatty acids, chelating agents, bile salts or mixtures thereof; capric acid, oleic acid and their monoglycerides, alkyl sulfates, sodium lauryl sulfate, sodium dodecyl sulfate, polysorbate 80, citric acid and phytic acid.

8. The controlled release pellet as defined in claim 7, wherein the surfactant is sodium lauryl sulphate.

9. The controlled release pellet as defined in claim 1, wherein the controlled release coating comprises a water insoluble or slightly water permeable polymer.

10. The controlled release pellet as defined in claim 9, wherein the water insoluble film-forming polymer is ethylcellulose.

11. The controlled release pellet as defined in claim 1, wherein the controlled release coating further comprises a pore forming agent.

12. The controlled release pellet as defined in claim 11, wherein the pore forming agent is a pH dependent material.

13. The controlled release pellet as defined in claim 12, wherein the pH dependent material is selected from the group consisting cellulose acetate phthalate, hydroxypropyl methylcellulose phthalate, polyvinylacetate phthalate, methacrylic acid copolymer, shellac, hydroxypropyl methylcellulose succinate, cellulose acetate trimellitate, hypromellose phthalate and mixtures thereof.

14. The controlled release pellet as defined in claim 13, wherein said enteric polymer is hypromellose phthalate.

15. The controlled release pellet as defined in claim 1, wherein the controlled release coating further comprises a plasticizer.

16. The controlled release pellet as defined in claim 15, wherein the plasticizer is selected from the group consisting of polyethylene glycol, propylene glycol, glycerol, triacetin, dimethyl phthalate, diethyl phthalate, dibutyl phthalate, dibutyl sebacate, triethyl citrate, acetyltriethyl citrate, tributyl citrate, triethyl acetyl citrate, castor oil, poloxamer and varying percentages of acetylated monoglycerides.

17. The controlled release pellet as defined in claim 16, wherein said plasticizer is acetyltributyl citrate.

18. An oral pharmaceutical tablet that comprises the controlled release pellet as defined in claim 1.

19. An oral pharmaceutical capsule that comprises the controlled release pellet as defined in claim 1.

20. A tablet as defined in claim 18 that further comprises and immediate release amount of tamsulosin.

21. A capsule as in claim 19 that further comprises an immediate release amount of tamsulosin.
